(19) United States
(12) Patent Application Publication
(10) Pub. No.: US 2009/0324760 A1
(43) Pub. Date: Dec. 31, 2009

(54) Hair tonic

(76) Inventors: Aya Komiya, Odawara-shi (JP); Kazuto Hamada, Odawara-shi (JP); Miki Taniguchi, Odawara-shi (JP)

Correspondence Address: MATHEWS, SHEPHERD, MCKAY, & BRUNEAU, P.A., 29 THANET ROAD, SUITE 201, PRINCETON, NJ, 08540, US

(21) Appl. No.: 11/919,311

(22) PCT Filed: Apr. 5, 2006
(86) PCT No.: PCT/EP2006/003080

§ 371 Date: Sep. 11, 2009

(30) Foreign Application Priority Data
Apr. 28, 2005 (JP) 2005-132045

(51) Int. Cl. A61K 36/63; A61K 31/7028

(52) U.S. Cl. 424/777; 514/35

(57) ABSTRACT

The present invention relates to hair tonic compositions containing acteoside of formula (I)

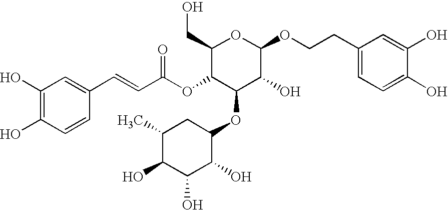

as an active ingredient. The hair tonic composition of the invention has an excellent hair-growth promoting effect and it can be used to prevent alopecia; furthermore, it has a pleasant skin feeling.

### FIELD OF THE INVENTION

The present invention relates to hair tonic compositions for the cosmetic treatment of hair-loss and alopecia.

### BACKGROUND OF THE INVENTION

Alopecia is deemed to be caused by several factors, such as defective circulation, decreased activity of the hair matrix and hair papilla cells and also hormonal unbalance. Aging and stress also play a major role and nowadays the risks of suffering from alopecia are high, due to the increase in the average age and the rhythms of modern life. However, alopecia causes have not yet been fully elucidated. The hair tonic compositions presently available on the market contain active ingredients having hair-nourishment and hair-growing effect, for example scalp circulation promoters such as pepper tincture, Swertia herb extract, ginseng extract and nicotinic acid derivatives; skin function promoters such as cepharanthine; melamine synthetic catalysts such as acetylcholine derivatives; keratolytic drugs such as salicylic acid and resorcin and anti-inflammatory agents such as glycyrrizinate and Lithospermum root extract. However, since in some cases these drugs cause cutaneous stimulation, their amount must be kept to a minimum, thus reducing their and hair-nourishment, hair-loss preventing and scurf preventing effect. Therefore, there is still the need for more effective hair tonic compositions.

Acteoside is a compound contained in a number of plants and it possesses different physiological activities; it has been disclosed as an anti-oxidant (J. Agric. Food Chem. 49:52-54, 2004), UV-absorber (JP-A No. 08-119842), 5-lipoxygenase inhibitor (JP-A No. 08-59685), allergy and adult disease preventive food (JP-A No. 61-291525) and discoloration inhibitor (JP-A No. 14-173608).

### DESCRIPTION OF THE INVENTION

It has now been found that acteoside is also endowed with an excellent hair-nourishing effect and that it can be used to promote hair-growth and prevent alopecia. Accordingly, the present invention relates to hair tonic compositions containing, as an active ingredient, acteoside of formula (I).

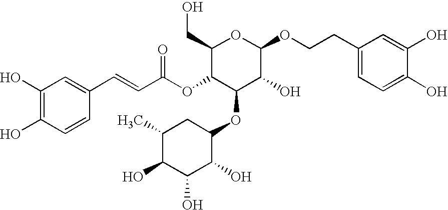

A second embodiment of the invention relates to hair tonic compositions comprising vegetation water obtained by pressing olive fruits.

A third embodiment of the invention relates to hair tonic compositions comprising a solid and/or aqueous phase obtained by pressing olive fruits.

A fourth embodiment of the invention relates to hair tonic compositions comprising an extract obtained by extracting a solid and/or aqueous phase obtained by pressing olive fruits with water and/or an organic solvent.

The hair tonic compositions of the invention have an excellent hair-growth promoting effect and also give a pleasant skin feeling.

Acteoside is contained in a number of plants such as the Oleaceae family, Lamiaceae family, Gesneriaceae family, Orobanchaceae family and Piperaceae and can be obtained from any one of these plants by extraction and purification. As an alternative, commercially available acteoside may be used. The fruits of the Oleaceae family, i.e. olives (Olea europaea L.) are relatively easy to find and are a useful source of acteoside. The fruits can be used irrespective of their production areas, species and intended use. However, the Coratina variety is particularly preferred due to its high acteoside content.

Vegetation water obtained by pressing olive fruits, or a solid and/or aqueous phase obtained by pressing olive fruits is preferably used as the acteoside-based hair tonic.

“Vegetation water” refers to the aqueous solution obtained by pressing olive fruits in ordinary pressing processes for the obtainment of olive oil from olive fruits. The solid phase isolated in the pressing process of olive fruits is a precipitate which may also partially include the liquid phase. The aqueous phase is obtained after olive oil is removed by means of, for instance, centrifugation.

The vegetation water, or the solid and/or aqueous phase can be used as such. However, the lipid or fibrous ingredients and seed shells therein contained are preferably removed by filtration and centrifugation.

In order to inhibit bacterial contamination and foul smell, hydrophilic alcohols and polyhydric alcohols such as ethyl alcohol, isopropyl alcohol, 1,3-butylene glycol and propylene glycol are preferably added so as to reach a final concentration ranging from 5 to 80% by weight, in particular from 10 to 40% by weight, followed by filtration and centrifugation.

The solid and/or aqueous phase obtained by pressing olive fruits are preferably extracted with water and/or an organic solvent. Organic solvents used in the extraction are hydrophilic alcohols and polyhydric alcohols such as ethyl alcohol, isopropyl alcohol, 1,3-butylene glycol and propylene glycol or mixtures thereof.

The vegetation water, the solid and/or aqueous phase and the extract can be used as such. If necessary, they can be concentrated and dried to a solid. Furthermore, in order to increase the acteoside content, they can be purified according to conventional procedures.

The acteoside content in the hair tonic composition of the invention is an amount that exhibit a hair-growth promoting effect, preferably 0.001% by weight or more of the total weight of the hair tonic. When the content is less than 0.001%, in some cases the hair-growth promoting effect is not sufficient. Furthermore, when the hair tonic of the invention is mixed with other compositions or used in various dosage formulations, the acteoside content is preferably 0.01% or more, more preferably 0.1% or more, to provide adequate bioavailability.

The amount of hair tonic is determined according to its acteoside content; however, the acteoside content in the hair tonic composition is preferably in the range of 0.001 to 10.0%, more preferably in the range of 0.001 to 5.0%. When the amount of acteoside is less than 0.001%, in some cases the alleged effect is not achieved. When it exceeds 10.0%, in some cases the increase in the effect is not as high as expected, the skin feeling may not be pleasant and side-effects may arise.

When a circulation promoter is also contained in the hair tonic composition of the invention, the hair-growth effect is further increased. When applied to the skin, circulation promoters improve blood circulation; their use is not subject to particular restrictions, since they do not cause safety problems in external preparations and their mechanism of action is not questioned. Specifically, circulation promoters that have been used so far comprise nicotinic acid derivatives such as benzyl nicotinate, tocopherol nicotinate, beta-buthoxyethyl nicotinate and nicotinamide; minoxidil and derivatives thereof and analogs thereof; cepharanthine; vitamin E and derivatives thereof; gamma-orizanol; alkoxycarbonyl pyridine-N-oxide; capronium chloride; acetylcholine and derivatives thereof; peripheral circulation disorder drugs (prostaglandin, carbaciclin, papaverine, cyclandelate, cinnarizine, nylidrin and kallikrein); calcium antagonists; phosphodiesterase inhibitors; nitrite compounds; adenosine action promoting vasodilators and hydrazine. One or more circulation promoters can be added to the hair tonic compositions of the invention. In the hair tonic composition according to the invention, the circulation promoter is preferably contained in the range of 0.001 to 5.0% of the total amount of the hair tonic composition, more preferably in the range of 0.01 to 2.0%. When the amount of circulation promoter is higher than 5.0%, there may be undesired side effects such as skin stimulation etc.

The hair tonic composition of the invention can be formulated in various forms; for instance, as dosage forms that are directly applied to the scalp and hair such as hair tonic, hair lotion, hair cream, shampoo, rinses, hair foams, hair gel and air-sol, which can be manufactured according to conventional procedures.

The hair tonic compositions of the invention, in addition to the aforementioned ingredients, can also comprise, if necessary, further ingredients generally used in cosmetics, external preparations and drugs, such as oils, wetting agents, thickeners, dyes, perfumes, germicides, antiseptics, UV-absorbers, solvents, water, keratolytic drugs, anti-inflammatory medications, anti-androgenic agents, hair tonics, anti-oxidants, refreshing agents, crude drug extracts and vitamins.

### Examples

The invention will be now described in more detail by means of some examples.

### Example 1

### Preparation of Crude Acteoside from Olives

Six kilograms of Coratina olive fruits were pressed and added with a hydroethanolic solution and the aqueous phase was extracted twice. The resulting extract was concentrated and 300 g of a dry solid was obtained. 250 g of the solid were extracted with 5.6 L of methanol; methanol was subsequently evaporated off under reduced pressure to afford 210 g of a methanol extract. The methanol extract was then dissolved in purified water up to 14.4 L and loaded onto a column filled with a synthetic styrenic adsorbent (Trade name: Amberlite XAD1180 manufactured by Organo Corporation). After washing with purified water, the column was eluted with methanol at various concentrations. The fraction containing 50% methanol were evaporated under pressure and freeze-dried. 25.05 g of “acteoside crude product” (42.0% by weight of acteoside) was obtained.

### Examples 2 and 3

### Evaluation of the Hair-Growth Promoting Effect

The hair-growth promoting effect of the hair tonic of the invention was evaluated according to the following hair-growth test. Commercially available acteoside (manufactured by ChromaDex Corp.) or the crude acteoside according to example 1 was dissolved in a 55% wt aqueous ethanol solution. Comparative example 1 was carried out using this aqueous ethanol solution as the reference.

Test Method

C3H mice (males of 8-week age, average weight 35 g, a group of 10 mice) were used as test animals. Their back (2 cm×4 cm) was shaved to prepare a test area and from the following day onwards, each test sample (0.2 mL per mouse) was applied twice a day(morning and evening) for two weeks. On the sixteenth day, a picture of the test area was taken with a video camera, and hair-growth was measured with an image analyzer. The hair-growth promoting effect was evaluated as hair-growth rate (%) calculated according to the equation reported below.

[Hair-growth rate (%)]=[area of the hair-growth portion]/[test area]×100

Results

The results of the hair-growth test, reported in table 1, show that the hair tonic of the invention has an outstanding hair growth promoting effect.

TABLE 1

| | | Sample | Hair growth |
|---|---|---|---|
| | | concentration | rate |
| | Sample | (% by weight) | (%) |
| Example 2 | Acteoside (manufactured | 0.1 | 61.05 |
| | by ChromaDex Corp.) | 0.2 | 65.02 |
| Example 3 | Crude | 0.2 | 62.94 |
| | acteoside(containing 42% | 0.5 | 64.96 |
| | of acteoside) | | |
| Comparative | (aqueous ethanol | — | 46.49 |
| example 1 | solution) | | |

### Examples 4 to 7

### Hair Growth and Skin Feeling Evaluation on Humans

Hair tonic compositions containing the hair tonic of the invention were prepared according to the prescriptions reported in Table 2, and the hair growth and skin feeling were evaluated according to the following test.

Test Method

Samples were applied daily (twice a day, morning and evening) for six months to 20 subjects suffering from male type alopecia. Thereafter, the subjects were asked to fill in questionnaires on the hair-growth effect and the alopecia preventing effect. The hair-growth effect was deemed to be shown when the patients answered that “fuzzy hair became bristly” and the alopecia preventing effect was deemed to be shown when the patients answered that “hair loss decreased”. The skin feeling test consisted in asking ten further subjects if, when applying 5 g of the sample on the scalp, they experienced any sticky feeling. The result of the feeling test upon use is expressed as the number of the subjects who answered in the positive.

TABLE 2

| | Amounts (% by weight) | | | | |
|---|---|---|---|---|---|
| | Exam- | Exam- | Exam- | | Comparative |
| Ingredient | ple 4 | ple 5 | ple 6 | Example 7 | Example 2 |
| Acteoside | 0.1 | 0.2 | — | — | — |
| (manufactured | | | | | |
| by ChromaDex | | | | | |
| Corp.) | | | | | |
| crude Acteoside | — | — | 0.2 | 0.5 | — |
| (Example 1) | | | | | |
| Polyoxtethylene | 0.5 | 0.5 | 0.5 | 0.5 | 0.5 |
| (60) sorbit | | | | | |
| tetraoleate | | | | | |
| Ethanol | 98.0 | 98.0 | 98.0 | 98.0 | 98.0 |
| Pure water | Balance | Balance | Balance | Balance | Balance |

Table 3 reports the results of the hair growth and feeling evaluation tests. As it can be seen from the table, the hair tonic composition containing the hair tonic of the invention proved excellent in both tests.

TABLE 3

| | | | | | Comparative |
|---|---|---|---|---|---|
| Evaluation | Example 4 | Example 5 | Example 6 | Example 7 | Example 2 |
| Hair | | | | | |
| growth | | | | | |
| test | | | | | |
| Hair- | 17 | 19 | 17 | 18 | 7 |
| growth | | | | | |
| effect | | | | | |
| Alopecia | 18 | 17 | 16 | 17 | 9 |
| preventing | | | | | |
| effect | | | | | |
| Skin | 2 | 1 | 1 | 2 | 5 |
| feeling | | | | | |

The invention can provide various kinds of hair tonic compositions having a hair growth effect; some examples are reported hereinbelow.

### Example 8

### Tonic

| | | Blending amount (% by |
|---|---|---|
| Ingredients | | weight) |
| (1) | 95% ethanol | 90.0 |
| (2) | Acteoside (manufactured by ChromaDex | 0.3 |
| | Corp.) | |
| (3) | Benzyl nicotinate | 0.05 |
| (4) | Acetic acid α-DL-tocopherol | 0.5 |
| (5) | Propylene glycol | 1.0 |
| (6) | Polyethylene glycol 200 | 1.0 |
| (7) | Perfume | 0.1 |
| (8) | Colors | Quantum sufficit |
| (9) | Pure water | Balance |

Preparation

Ingredients (2) to (7) were dissolved in (1), followed by addition of (8) and (9); the resulting mixture was stirred, thereby obtaining a transparent liquid tonic.

### Example 9

### Hair Growth Lotion

| Ingredients | | Blending amount (%) |
|---|---|---|
| (1) | 95% ethanol | 80.0 |
| (2) | Crude acteoside according to example 1 | 0.5 |
| (3) | Nicotinamide | 0.5 |
| (4) | Cepharanthine | 0.01 |
| (5) | Dipropylene glycol | 2.0 |
| (6) | Disodium phosphate | 0.08 |
| (7) | Monopotassium phosphate | 0.02 |
| (8) | L-menthol | 0.3 |
| (9) | Perfume | 0.05 |
| (10) | Hydrogenated bisabolol | 0.5 |
| (11) | Pure water | Balance |

Preparation

Ingredients (2) to (5), (8), (9) and (10) were dissolved in (1), then added with a solution of (6) and (7) in (11) and stirred, to give and a transparent liquid hair tonic lotion.

### Example 10

### Aerosol Hair Growth Composition

| | |
|---|---|
| Ingredients | Amount (%) |

Undiluted solution

| | | |
|---|---|---|
| (1) | 95% ethanol | 50.0 |
| (2) | Glycyrrhizinic acid | 0.1 |
| (3) | Crude acteoside according to example 1 | 0.5 |
| (4) | Swertia herb extract | 2.0 |
| (5) | Sodium lauryl sulfate | 0.1 |
| (6) | Polyoxyethylene (40) hydrogenated castor | 0.5 |
| | oil | |
| (7) | Lactic acid | Quantum sufficit |
| (8) | Sodium lactate | Quantum sufficit |
| (9) | Perfume | Quantum sufficit |
| (10) | Colors | Trace |
| (11) | Pure water | Balance |

<Final composition>

| | | |
|---|---|---|
| (12) | Undiluted solution | 50.0 |
| (13) | Liquefied petroleum gas | 50.0 |

Preparation—Ingredients (1) to (11) were mixed and dissolved to prepare an undiluted solution which was filled into a canister. Liquefied petroleum gas was added after fitting the canister with a valve.

### Example 11

### Emulsion Hair Tonic Composition

| | |
|---|---|
| Ingredient | Blending amount (%) |

<Phase A>

| | | |
|---|---|---|
| (1) | Crude acteoside according to example 1 | 0.5 |
| (2) | Polyoxyethylene (40) hydrogenated castor | 2.0 |
| | oil | |
| (3) | Glycerin | 10.0 |
| (4) | Dipropylene glycol | 10.0 |
| (5) | 1,3-butylene glycol | 5.0 |
| (6) | Polyethylene glycol 1500 | 5.0 |

<Phase B>

| | | |
|---|---|---|
| (7) | Cetyl isooctanate | 10.0 |
| (8) | Squalane | 5.0 |
| (9) | Vaseline | 2.0 |
| (10) | Propylparaben | 2.0 |

<Phase C>

| | | |
|---|---|---|
| (11) | Carboxy vinyl polymer (1% aqueous | 30.0 |
| | solution) | |
| (12) | Sodium hexametaphosphate | 0.03 |
| (13) | Pured water | 8.0 |

<Phase D>

| | | |
|---|---|---|
| (14) | Pure water | 5.0 |

<Phase E>

| | | |
|---|---|---|
| (15) | Potassium hydroxide | 0.12 |
| (16) | Pure water | Balance |

Preparation

Phases A and B were heated and dissolved separately at 70° C., then mixed and processed with a homo-mixer to obtain a gel. Phase D was gradually added to the gel and dispersed with a homo-mixer. Phases C and E were dissolved separately, added to the gel dispersion and emulsified with a homo-mixer to give an emulsion.

1. A hair tonic composition comprising as an active ingredient, acteoside of formula (I): 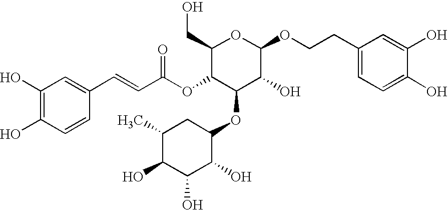
in admixture with physiologically acceptable excipients.

2. A hair tonic composition comprising vegetation oil obtained by pressing olive fruits in admixture with physiologically acceptable excipients.

3. A hair tonic composition comprising a solid and/or an aqueous phase obtained by pressing olive fruits in admixture with physiologically acceptable excipients.

4. A hair tonic composition comprising an extract obtained by extracting the solid and/or aqueous phase obtained by pressing olive fruits with water and/or an organic solvent in admixture with physiologically acceptable excipients.

5. A composition according to claim 1 wherein the amount of acteoside ranges from 0.001 to 10.0%.

6. A composition according to claim 5 wherein the amount of acteoside ranges from 0.001 to 5%.

7. A hair tonic composition according to claim 1 further comprising one or more circulation promoters.

8. A hair tonic composition according to claim 7 wherein the circulation promoter is selected from benzyl nicotinate, tocopherol nicotinate, beta-buthoxyethyl nicotinate and nicotinamide; minoxidil; cepharanthine; vitamin E; gamma-orizanol; alkoxycarbonyl pyridine-N-oxide; capronium chloride; acetylcholine; prostaglandin, carbaciclin, papaverine, cyclandelate, cinnarizine, nylidrin and kallikrein; calcium antagonists; phosphodiesterase inhibitors; nitrite compounds; adenosine action promoting vasodilators and hydrazine.

9. The hair-tonic composition according to claim 8 wherein the amount of circulation promoter ranges from 0.001 to 5% of the composition.

10. The hair-tonic of claim 9 wherein the amount of circulation promoter ranges from 0.001 to 2% of the composition.

11. The use of the compositions of claim 1 for the cosmetic treatment of alopecia and promoting hair-growth.
